(19) United States
(12) Patent Application Publication
(10) Pub. No.: US 2006/0020054 A1
(43) Pub. Date: Jan. 26, 2006

(54) Aromatic accessory having pack containing aromatic component

(76) Inventor: Byung-Ok An, Seoul (KR)

Correspondence Address: LADAS & PARRY LLP, 224 SOUTH MICHIGAN AVENUE, SUITE 1600, CHICAGO, IL, 60604, US

(21) Appl. No.: 11/185,195

(22) Filed: Jul. 20, 2005

(30) Foreign Application Priority Data
Jul. 21, 2004 (KR) KR20-2004-0020831

(51) Int. Cl. A61L 9/04

(52) U.S. Cl. 523/102

(57) ABSTRACT

An aromatic accessory having a pack containing fragrances is provided. The pack in the aromatic accessory is an urethane pack made of a transparent polyurethane material and having a number of microscopic pores which pass gaseous components and block liquid components. The urethane pack is made of matured urethane and is filled with an aromatic component in which oil fragrance of 20 to 50 weight % and transparent paraffin oil of 50 to 80 weight % are mixed with each other. A shape forming member on which a variety of characters or logoes are printed, and which plays a role of maintaining shape of the urethane pack, is inserted into the urethane pack. The aromatic accessory is hung on a cellular phone or personal ornamental article, and carried portably. Accordingly, fragrances are persistently emitted through microscopic pores in the matured urethane pack and simultaneously advertisement, publicity, publication relations, and esthetic appreciation can be obtained by symbols, characters, shapes and appearances printed on the shape forming members. An extension of period of and an amount of emission of aroma can be controlled according to an amount of fragrances or a degree of aging fragrances which are sealed in the matured urethane pack. Aromatic materials which can perform an antibiotic function are filled in the urethane pack according to the kind of the aroma as desired, to thus provide an effect of preventing a parasite of bacteria.

### BACKGROUND OF THE INVENTION

1. Field of the Invention

The present invention relates to an aromatic accessory, and more particularly, to an aromatic accessory having a pack containing aromatic component in which the pack in the aromatic accessory is an urethane pack made of a transparent polyurethane material and having a number of microscopic pores which pass gaseous components and block liquid components, the urethane pack is made of matured urethane and is filled with an aromatic component in which oil fragrance of 20 to 50 weight % and transparent paraffin oil of 50 to 80 weight % are mixed with each other, and a shape forming member on which a variety of characters or logoes are printed, and which plays a role of maintaining shape of the urethane pack, is inserted into the urethane pack.

2. Description of the Related Art

Conventional aromatic accessories made of transparent polyurethane material and having a number of microscopic pores which pass gaseous components and block liquid components, are filled with aromatic components and have a low transmissivity of moisture and a low transmissivity of oxygen and gas. The aromatic components are not matured differently from the present invention. Accordingly, microscopic pores are small in numbers to thus emit a small amount of fragrances. As a result, a sufficient effect of emission of fragrances cannot be obtained on a sense-of-smell basis. For these reasons, a range of emission of aroma is limited.

The conventional aromatic accessories emit fragrances externally through microscopic pores, in which aqueous aromatic fragrances mixed with flavors and staining materials are injected in respective urethane packs made of flexible polyurethane materials. They are hung on key-rings, cellular phones, or various ornamental articles and used as fragrances or accessories.

The urethane pack in the conventional aromatic accessories are filled with aqueous fragrances mixed with staining materials. In this case, the urethane pack can be easily filled with aqueous fragrances. However, if the urethane pack is broken or damaged, persons who carry the conventional aromatic accessories portably can be severely polluted by the staining materials. Also, the conventional aromatic accessories can be easily deformed due to thin-film flexible urethane packs during filling or using aromatic components, in which circumferential portions of the urethane packs are contracted or twisted. Accordingly, original shapes cannot be maintained.

### SUMMARY OF THE INVENTION

To solve the above problems, it is an object of the present invention to provide an aromatic accessory which can increase a range of emission of aroma as well as an amount of emission of aroma according to aged microscopic pores, to accordingly persist aromatic emission for long over a wide range, and which can always maintain an original shape during production of products with a shape forming member.

It is another object of the present invention to provide an aromatic accessory in which microscopic magnetic materials are coated on one surface of the shape forming member, so that the aromatic accessory can be attached on home appliances such as refrigerators or steel-made doors, and thus can be conveniently used.

To accomplish the above object of the present invention, there is provided an aromatic accessory comprising: a matured urethane pack made of flexible transparent polyurethane and having a number of microscopic pores which pass gaseous components and block liquid components; an aromatic component filled into the urethane pack, in which oil fragrances and transparent paraffin oil are mixed with each other; and a shape forming member made of a rigid material which plays a role of maintaining shape of the urethane pack.

### BRIEF DESCRIPTION OF THE DRAWINGS

The above and other objects and advantages of the present invention will become more apparent by describing the preferred embodiment thereof in more detail with reference to the accompanying drawings in which:

FIG. 1 is a perspective view showing an aromatic accessory according to the present invention; and

FIG. 2 is a schematic cross-sectional view showing the aromatic accessory of FIG. 1.

### DETAILED DESCRIPTION OF THE INVENTION

An aromatic accessory according to a preferred embodiment of the present invention will be described below with reference to the accompanying drawings.

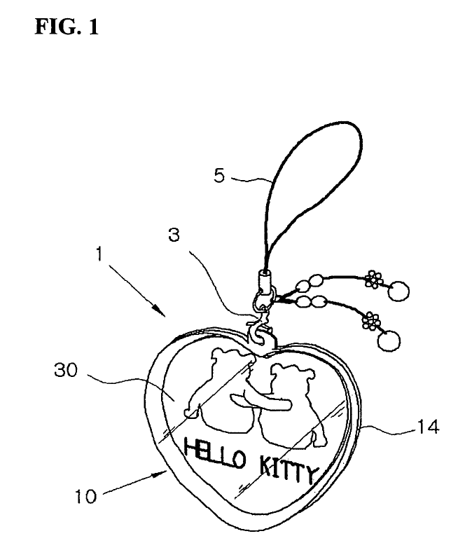

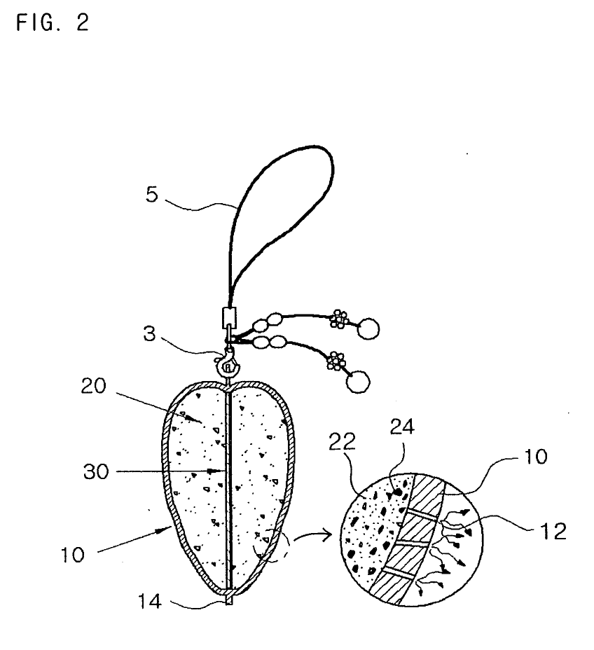

Referring to FIGS. 1 and 2, an aromatic accessory according to the present invention includes a pack containing aromatic components. The pack in the aromatic accessory is an urethane pack 10 made of a flexible, transparent polyurethane material and having a number of microscopic pores 12 which pass gaseous components and block liquid components. The urethane pack is filled with an aromatic component 20 in which oil fragrance of 20 weight % to 50 weight % and transparent paraffin oil of 50 weight % to 80 weight % are mixed with each other.

The urethane pack 10 is made of matured urethane. A shape forming member 30 made of a rigid material, on which a variety of symbols, characters or logoes such as shapes and forms are printed, and which plays a role of maintaining shape of the urethane pack 10, is inserted into the urethane pack 10. In order to prevent a circumferential portion 14 of the urethane pack 10 from being contracted or deformed, the shape forming member 30 is formed in size smaller than the circumferential portion 14 of the urethane pack 10. An amount of fragrances to be filled in the urethane pack 10 can be controlled according to the size of the shape forming member 30. Also, the shape forming member 30 should have a sufficient rigidity in order to maintain the shape of the urethane pack 10.

In FIGS. 1 and 2, a reference numeral 3 denotes a connection ring and a reference numeral 5 denotes a hanging string.

Meanwhile, although one side of the shape forming member 30 is not shown in the drawings, microscopic magnetic materials are coated on one surface of the shape forming members in the aromatic accessory. Accordingly, the aromatic accessory can be attached on home appliances such as refrigerators or steel-made doors, and thus can be conveniently used.

As described above, an aromatic component 20 is sealed in the urethane pack 10 applied in the aromatic accessory 1 according to the present invention, in which oil fragrance 22 of 20 weight % to 50 weight % and transparent paraffin oil 24 of 50 weight % to 80 weight % are mixed with each other. The aromatic component 20 emits via the microscopic pores 12 formed on the urethane pack 10. Thus, aroma can be emitted for six through twelve months or more according to a capacity of the aromatic component 20. An extension of period of and an amount of emission of aroma can be controlled according to an amount of fragrances or a degree of aging fragrances which are sealed in the matured urethane pack 10.

Meanwhile, since the aromatic component 20 obtained by mixing the oil fragrance 22 and the transparent paraffin oil 24 is of an oily feature, the circumferential portion 14 of the urethane pack 10 can be easily fused or melted during putting the aromatic component 20 in the urethane pack 10 and sealing it. Also, the aromatic component 20 is not leaked since perfect sealing is accomplished even after fusing or melting.

Also, both side surfaces of the shape forming member 30 functions as a shape reinforcing member playing a role of a support which maintains the shape of the urethane pack 10. As well, user's desired printing matters are printed on either side surface of the shape forming member 30 to function to provide advertisement, publicity, publication relations, and esthetic appreciation.

Also, in the case that an inherent fragrance extracted from DNA of a particular person is mixed in the aromatic component 20, a unique fragrance can emit.

As described above, in the case of the aromatic accessory according to the present invention, the aromatic component obtained by mixing the oil fragrance and the transparent paraffin oil is injected into the matured urethane pack, and the shape forming member on both side surfaces of which a variety of symbols, characters or logoes such as shapes and forms are printed, and on one side surface of which a microscopic magnetic material is coated is inserted into the urethane pack. The aromatic accessory is hung on a cellular phone or personal ornamental article, and carried portably. Accordingly, fragrances are persistently emitted through microscopic pores in the matured urethane pack and simultaneously advertisement, publicity, publication relations, and esthetic appreciation can be obtained by symbols, characters, shapes and appearances printed on the shape forming members. An extension of period of and an amount of emission of aroma can be controlled according to an amount of fragrances or a degree of aging fragrances which are sealed in the matured urethane pack. Aromatic materials which can perform an antibiotic function are filled in the urethane pack according to the kind of the aroma as desired, to thus provide an effect of preventing a parasite of bacteria.

As described above, the present invention has been described with respect to a particularly preferred embodiment. However, the present invention is not limited to the above embodiment, and it is possible for one who has an ordinary skill in the art to make various modifications and variations, without departing off the spirit of the present invention.

What is claimed is:

1. An aromatic accessory comprising:
a matured urethane pack made of flexible transparent polyurethane and having a number of microscopic pores which pass gaseous components and block liquid components;
an aromatic component filled into the urethane pack, in which oil fragrances and transparent paraffin oil are mixed with each other; and
a shape forming member made of a rigid material which plays a role of a support maintaining shape of the urethane pack.

2. The aromatic accessory according to claim 1, wherein, in which advertisement, publicity, publication relations, and esthetic appreciation can be obtained by symbols, characters, shapes and appearances printed on the shape forming members

3. The aromatic accessory according to claim 1, wherein a magnetic material is coated on one side surface of the shape forming member.

4. The aromatic accessory according to claim 1, wherein the shape forming member is formed in size smaller than the circumferential portion of the urethane pack to maintain shape of the urethane pack, to thus adjust an amount of the aromatic component according to the size of the shape forming member during filling the aromatic component in the urethane pack.

5. The aromatic accessory according to claim 1, wherein the aroma can be emitted according to a capacity of the aromatic component.

6. The aromatic accessory according to claim 5, wherein since the aromatic component obtained by mixing the oil fragrance and the transparent paraffin oil is of an oily feature, the circumferential portion of the urethane pack can be easily fused or melted during putting the aromatic component in the urethane pack and sealing it.
